(19) United States
(12) Patent Application Publication
(10) Pub. No.: US 2010/0041911 A1
(43) Pub. Date: Feb. 18, 2010

(54) Process For The Production Of (Alkoxycarbonylamino)alkyl Sulfonates

(75) Inventors: David Cladingboel, Leicestershire (GB); Adam Herring, Leicestershire (GB); Rhona Sinclair, Leicestershire (GB)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/993,029

(22) PCT Filed: Jun. 12, 2006
(86) PCT No.: PCT/SE2006/000694

§ 371 Date: Dec. 19, 2007

(30) Foreign Application Priority Data
Jun. 20, 2005 (SE) 0501429-5
Dec. 15, 2005 (SE) 0502770-1

(51) Int. Cl. C07C 303/02

(52) U.S. Cl. 558/49

(57) ABSTRACT

There is provided a process for the preparation of a compound of formula I, which process comprises: (a) reaction of a compound of formula II, HO-D-NH₂ II with a compound of formula III, followed by (b) reaction of the intermediate of formula IV thereby formed, IV with base and a compound of formula V, R²S(O)₂L² V, wherein the intermediate of formula IV is not isolated, and wherein D, R¹, R², L¹ and L² have meanings given in the description.

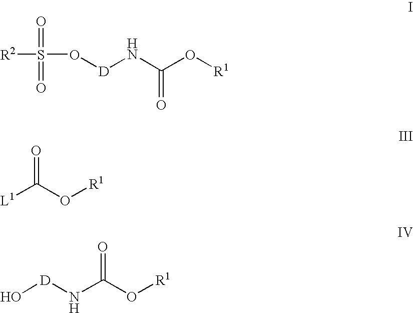

### FIELD OF THE INVENTION

There is provided a novel process for the preparation of a (alkoxycarbonylamino)-alkyl sulfonate, which compound may be employed in the synthesis of a range of oxabispidines that bear an (alkoxycarbonylamino)alkyl substituent.

### BACKGROUND AND PRIOR ART

Compounds comprising alkylene groups having a leaving group at one end and an alkoxycarbonylamino substituent at the other end are useful intermediates in the preparation of certain bioactive molecules (e.g. those bearing (alkoxycarbonyl-amino)alkyl substituents).

International patent applications WO 01/028992 and WO 02/083690 disclose oxabispidines bearing 2-(alkoxycarbonylamino)ethyl substituents, which compounds are indicated as being useful in the treatment of cardiac arrhythmias.

In WO 01/028992, the relevant compounds are prepared using an intermediate having a halide leaving group (2-(tert-butyloxycarbonylamino)ethyl bromide). In contrast, WO 02/083690 describes the use of a sulfonate-containing intermediate (2-(tert-butoxycarbonylamino)ethyl 2,4,6-trimethylbenzenesulfonate) for the preparation of the relevant compounds. This reagent is described in WO 02/083690 as being prepared from 2-(tert-butoxycarbonylamino)ethanol.

However, there is no disclosure or suggestion in any of the above-mentioned documents of the synthesis of an (alkoxycarbonylamino)alkyl sulfonate in two steps and without isolation of intermediates (i.e. in a “one-pot” process) directly from the corresponding aminoalkanol.

We have now surprisingly found that (alkoxycarbonylamino)alkyl sulfonate reagents may be prepared by way of such a “one-pot” process.

### DISCLOSURE OF THE INVENTION

There is provided a process for the preparation of a compound of formula I,

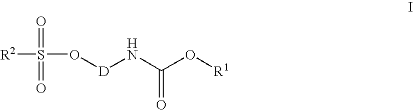

wherein D represents C₂₋₆ alkylene;
R¹ represents C₁₋₆ alkyl (optionally substituted by one or more substituents selected from —OH, halo, cyano, nitro and aryl), aryl or Het¹;
R² represents unsubstituted C₁₋₄ alkyl, C₁₋₄ perfluoroalkyl or phenyl, which latter group is optionally substituted by one or more substituents selected from C₁₋₆ alkyl, halo, nitro and C₁₋₆ alkoxy;
Het¹ represents a 4- to 14-membered heterocyclic group containing one or more heteroatoms selected from oxygen, nitrogen and/or sulfur, which heterocyclic group may comprise one, two or three rings and may be substituted by one or more substituents selected from oxo, halo, nitro, C₁₋₆ alkyl and C₁₋₁₆ alkoxy (which latter two groups are optionally substituted by one or more halo atoms); and
wherein each aryl group, unless otherwise specified, is optionally substituted;
provided that D does not represent 1,1-C₂₋₆ alkylene;
which process comprises:
(a) reaction of a compound of formula II,

HO-D-NH₂ II
wherein D is as hereinbefore defined, with a compound of formula III,

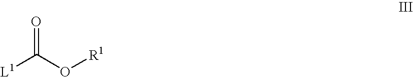

wherein L¹ represents a leaving group and R¹ is as defined above; followed by
   (b) reaction of the intermediate of formula IV thereby formed,

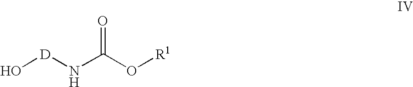

wherein D and R¹ are as hereinbefore defined, with base and a compound of formula V,

R²S(O)₂L² V
wherein L² represents a leaving group and R² is as defined above,
   and wherein the intermediate of formula IV is not isolated,
   which process is hereinafter referred to as “the process of the invention”.

By “not isolated”, we mean that the intermediate of formula IV is not actively separated from any unreacted reagents (i.e. the compounds of formulae II and III) or by-products formed after the formation of the compound of formula IV is substantially complete. In this respect, it is preferred that the process of the invention is performed as a “one-pot process”, i.e. where the two consecutive reactions are performed in the same reaction vessel. More preferably, the process is performed by completion of the reaction between the compounds of formulae II and III and then, without work-up, addition of base and the compound formula V to the resulting product mixture.

Alkylene groups as defined herein may be straight-chain or, when there is a sufficient number (i.e. a minimum of two) of carbon atoms, be branched-chain. Such alkylene chains may also be saturated or, when there is a sufficient number (i.e. a minimum of two) of carbon atoms, be unsaturated and/or interrupted by one or more oxygen and/or sulfur atoms. However, such alkylene groups are preferably saturated and not interrupted by any such heteroatoms. Alkylene groups may also be substituted by one or more halo atoms, but are nevertheless preferably not so substituted.

Unless otherwise specified, alkyl groups and alkoxy groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl and alkoxy groups may also be part cyclic/acyclic. Such alkyl and alkoxy groups may also be saturated or, when there is a sufficient number (i.e. a minimum of two) of carbon atoms, be unsaturated and/or interrupted by one or more oxygen and/or sulfur atoms. Unless otherwise specified, alkyl and alkoxy groups may also be substituted by one or more halo, and especially fluoro, atoms.

The term “aryl”, when used herein, includes C₆₋₁₃ aryl (e.g. C₆₋₁₀) groups. Such groups may be monocyclic, bicyclic or tricylic and, when polycyclic, be either wholly or partly aromatic. In this respect, C₆₋₁₃ aryl groups that may be mentioned include phenyl, naphthyl, 1,2,3,4-tetrahydronaphthyl, indanyl, indenyl, fluorenyl and the like. For the avoidance of doubt, the point of attachment of substituents on aryl groups may be via any carbon atom of the ring system.

Unless otherwise specified, aryl groups may be substituted by one or more substituents selected from —OH, cyano, halo, nitro, C₁₋₆ alkyl, C₁₋₆ alkoxy, —N(R^{3a})R^{3b}, —C(O)R^{3c}, —C(O)OR^{3d}, —C(O)N(R^{3e})R^{3f}, —N(R^{3g})C(O)R^{3h}, —N(R³ⁱ)S(O)₂R^{4a}, —S(O)₂N(R^{3j})R^{3k}, —S(O)₂R^{4b} and/or —OS(O)₂R^{4c}, (wherein R^{3a} and R^{3b} independently represent H, C₁₋₆ alkyl, or together represent C₃₋₆ alkylene, resulting in a four- to seven-membered nitrogen-containing-ring, R^{3c} to R^{3k} independently represent H or C₁₋₆ alkyl and R^{4a} to R^{4c} independently represent C₁₋₆ alkyl). When substituted, aryl groups are preferably substituted by between one and three substituents. For the avoidance of doubt, the point of attachment of aryl groups may be via any carbon atom of the ring system.

The term “halo”, when used herein, includes fluoro, chloro, bromo and iodo.

Compounds employed in or produced by the processes described herein (i.e. those involving the process of the invention) may exhibit tautomerism. The process of the invention therefore encompasses the use or production of such compounds in any of their tautomeric forms, or in mixtures of any such forms.

Similarly, the compounds employed in or produced by the processes described herein (i.e. those involving the process of the invention) may also contain one or more asymmetric carbon atoms and may therefore exist as enantiomers or diastereoisomers, and may exhibit optical activity. The process of the invention thus encompasses the use or production of such compounds in any of their optical or diastereoisomeric forms, or in mixtures of any such forms.

Abbreviations are listed at the end of this specification.

Preferred compounds of formula I include those in which:

D represents —(CH₂)₃— or, particularly, —(CH₂)₂—;
R¹ represents C₁₋₆ alkyl, particularly saturated C₁₋₆ alkyl;
R² represents phenyl, optionally substituted by one or more (e.g. one to three) substituents (e.g. one substituent) selected from C₁₋₃ alkyl (e.g. methyl), halo and nitro.

More preferred compounds of formula I include those in which:

R¹ represents secondary or tertiary C₃₋₅ alkyl, particularly saturated s- or t-C₄ alkyl;
R² represents halophenyl (e.g. 4-chlorophenyl) or, particularly, unsubstituted phenyl, methylphenyl (such as 4-methylphenyl) or trimethylphenyl (such as 2,4,6-trimethylphenyl).

Particularly preferred compounds of formula I include those in which:

R¹ represents tert-butyl;
R² represents 2,4,6-trimethylphenyl.

Specific compounds of formula I that may be mentioned include:
2-(tert-butyloxycarbonylamino)ethyl 2,4,6-trimethylbenzenesulfonate; and
3-(tert-butyloxycarbonylamino)propyl 4-chlorobenzenesulfonate.

Preferred compounds of formula II include those in which D represents —(CH₂)₃— (i.e. 3-amino-1-propanol) or, particularly, —(CH₂)₂— (i.e. 2-aminoethanol).

As stated above in respect of compounds of formula III, L¹ represents a leaving group. Suitable leaving groups that L¹ may represent include halo and, particularly, -X-R⁵, wherein:

X represents —O—, —O—C(O)O—, —O—N═C(CN)—, —O—N(R^{5a})C(O)O—, —O—P(O)(OR^{5b})—O— or —O—O—;
R⁵ represents C₁₋₆ alkyl (optionally substituted by one or more substituents selected from —OH, halo, cyano, —C(O)C₁₋₄ alkyl and aryl), Het² or aryl;
R^{5a} and R^{5b} independently represent H or C₁₋₆ alkyl (optionally substituted by one or more halo atoms); and
Het² represents a 4- to 14-membered heterocyclic group containing one or more heteroatoms selected from oxygen, nitrogen and/or sulfur, which heterocyclic group may comprise one, two or three rings and may be substituted by one or more substituents selected from oxo, halo, nitro and C₁₋₆ alkyl (which latter group is optionally substituted by one or more halo atoms).

More preferred compounds of formula III include those in which:

L¹ represents -X-R⁵;
X represents —O— or —O—C(O)O—;
R⁵ represents aryl or C₁₋₆ alkyl (e.g. saturated C₁₋₆ alkyl, such secondary or tertiary C₃₋₅ alkyl or, particularly, s- or t-C₄ alkyl).

Especially preferred compounds of formula III include those in which:

X represents —O—C(O)O—;
R⁵ represents tert-butyl.

Het (Het¹ and Het²) groups that may be mentioned include those containing 1 to 4 heteroatoms (selected from the group oxygen, nitrogen and/or sulfur) and in which the total number of atoms in the ring system are between five and fourteen. Het (Het¹ and Het²) groups may be fully saturated, wholly aromatic, partly aromatic and/or bicyclic in character. Heterocyclic groups that may be mentioned include 1-azabicyclo[2.2.2]octanyl, benzimidazolyl, benzisoxazolyl, benzodioxanyl, benzodioxepanyl, benzodioxolyl, benzofuranyl, benzofurazanyl, benzo-morpholinyl, 2,1,3-benzoxadiazolyl, benzoxazinonyl, benzoxazolidinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cirmolinyl, 2,3-dihydrobenzimidazolyl, 2,3-dihydrobenzo[b]furanyl, 1,3-dihydrobenzo-[c]furanyl, 2,3-dihydropyrrolo[2,3-b]pyridyl, dioxanyl, furanyl, hexahydro-pyrimidinyl, hydantoinyl, imidazolyl, imidazo[1,2-a]pyridyl, imidazo[2,3-b]-thiazolyl, indolyl, isoindolinyl, isoquinolinyl, isoxazolyl, maleimido, morpholinyl, oxadiazolyl, 1,3-oxazinanyl, oxazolyl, phthalazinyl, piperazinyl, piperidinyl, purinyl, pyranyl, pyrazinyl, pyrazolyl, pyridyl, pyrimidinyl, pyrrolidinonyl, pyrrolidinyl, pyrrolinyl, pyrrolo[2,3-b]pyridyl, pyrrolo[5,1-b]pyridyl, pyrrolo[2,3-c]pyridyl, pyrrolyl, quinazolinyl, quinolinyl, sulfolanyl, 3-sulfolenyl, 4,5,6,7-tetra-hydrobenzimidazolyl, 4,5,6,7-tetrahydrobenzopyrazolyl, 5,6,7,8-tetrahydrobenzo-[e]pyrimidine, tetrahydrofuranyl, tetrahydropyranyl, 3,4,5,6-tetrahydropyridyl, 1,2,3,4-tetrahydropyrimidinyl, 3,4,5,6-tetrahydropyrimidinyl, thiadiazolyl, thiazolidinyl, thiazolyl, thienyl, thieno[5,1-c]pyridyl, thiochromanyl, triazolyl, 1,3,4-triazolo[2,3-b]pyrimidinyl and the like.

Substituents on Het (Het¹ and Het²) groups may, where appropriate, be located on any atom in the ring system including a heteroatom. The point of attachment of Het groups may be via any atom in the ring system including (where appropriate) a heteroatom, or an atom on any fused carbocyclic ring that may be present as part of the ring system. Het (Het¹ and Het²) groups may also be in the N- or S-oxidised form.

Particular values of Het² that may be mentioned include quinolinyl (e.g. 8-quinolinyl), N-phthaliridyl and N-succinimidyl.

It is preferred that the process of the invention is performed in the presence of solvent. In this respect, the solvent is preferably an organic solvent or a mixture of organic solvents. Such solvents include di(C₁₋₆ alkyl)ethers (such as di(C₁₋₄ alkyl)ethers, e.g. diethyl ether), C₁₋₆ alkyl acetates (such as C₁₋₄ alkyl acetates, e.g. ethyl acetate), chlorinated hydrocarbons (e.g. chlorinated C₁₋₄ alkanes such as dichloromethane, chloroform, carbon tetrachloride and 1,2-dichloroethane), hexane, petroleum ether, and aromatic hydrocarbons, such as benzene and mono-, di- or tri-alkylbenzenes (e.g. mesitylene, xylene, or toluene). Particularly preferred organic solvents include C₁₋₂ alkanes, which groups are substituted with one or more chloro groups. In this respect, preferred solvents include chloroform, carbon tetrachloride, 1,2-dichloroethane and, particularly, dichloromethane.

It is particularly preferred that the same solvent system is employed for both steps of the two-part process of the invention (i.e. for steps (a) and (b) above).

In a particularly preferred embodiment of the invention, a catalyst is employed to enhance the reactivity of the sulfonylating reagent of formula V. In this embodiment, the catalyst may be added to the reaction mixture at any point, but particularly after the reaction between the aminoalcohol of formula II and the compound of formula III is substantially complete (i.e. at approximately the same time as the compound of formula V is added to the reaction mixture, and preferably immediately prior to the addition of the compound of formula V).

Such catalysts include tertiary amines (e.g. tri(C₁₋₃ alkyl)amines, pyridine and dimethylaminopyridine (DMAP)), optionally in the form of an acid addition salt (e.g. tri(C₁₋₃ alkyl)amine hydrohalide salts, such as trimethylamine hydrochloride; see Tetrahedron, 1999, 55(8), 2183-2192).

Preferably, the reaction between the aminoalcohol of formula II and the compound of formula III (step (a) above) is conducted at elevated (i.e. above ambient) temperature, such as from 20° C. or, preferably, 30° C. to reflux. For example, when dichloromethane is the solvent employed for this reaction, the reaction mixture may be heated to any temperature from 32° C. to reflux (e.g. to about 35° C.). In this embodiment, it is further preferred that a mixture of the aminoalcohol of formula II and dichloromethane is first heated to such a temperature before reaction is initiated by the addition of the compound of formula III.

In a particular embodiment of the invention, the compound of formula III is added to a mixture of reaction solvent (see above) and compound of formula II in neat (i.e. undiluted) form or, preferably, as a solution in, for example, the same solvent system in which the reaction with the aminoalcohol of formula II is conducted. In this embodiment, the compound of formula III is dissolved in from 2 to 8 (e.g. about 5) relative volumes of solvent and is added to a mixture of compound of formula II and from 4 to 12 (e.g. about 8) relative volumes of solvent.

The compound of formula III may added at any rate, but preferably at a rate in the range from 0.1 to 500 mmol per minute, such as about 6 mmol per minute.

After the compound of formula III has been mixed with the aminoalcohol of formula II, then the reaction may be stirred for any length of time, but preferably any time from 10 minutes to 4 hours, such as from 30 minutes to 2 hours (e.g. about 1 hour), or time sufficient to effect dissolution of any oily substance that may have previously formed.

The stoichiometric ratio of the aminoalcohol of formula II to the compound of formula III is preferably in the range from 2:1 to 1:2, a particular embodiment of which being about 1:1.

If necessary, the reaction between the aminoalcohol of formula II and the compound of formula III (step (a) above) is performed in the presence of base. However, it is preferred that this reaction step is performed in the absence of base.

For the reaction between the compounds of formulae IV and V (step (b) above), any suitable base may be employed. For example, when the reaction solvent is organic, then the base employed is preferably soluble in that organic solvent. Suitable bases therefore include tertiary amines, such as tertiary aromatic or heterocyclic amines or, particularly, tertiary aliphatic amines, such as tri-(C₁₋₆ alkyl)amines (e.g. trimethylamine and, particularly, triethylamine).

The quantity of base employed in the reaction between the compounds of formulae IV and V is preferably at least equimolar to the quantity of the aminoalcohol of formula II employed in the first step of the process of the invention. For example, the stoichiometric ratio of base to the aminoalcohol of formula II may be any value at or above 1:1, such as from 1:1 to 5:1, preferably from 11:10 to 5:2 (e.g. about 3:2).

When a tertiary amine acid addition salt is employed as a catalyst in the reaction between the compounds of formulae IV and V then the quantity employed may be (in comparison with the quantity of the aminoalcohol of formula II employed in step (a) above) any amount, such as from 0.1 to 1 molar equivalents (e.g. from 0.4 to 0.8 molar equivalents, such as about 0.5 or 0.7 molar equivalents). The skilled person will appreciate that, for optimum yield, the molar quantity of base minus the molar quantity of tertiary amine acid addition salt employed should be at least one molar equivalent (compared to the quantity of the compound of formula V employed in the second step).

When trimethylamine, or an acid addition salt thereof, is employed as a catalyst, the reaction between the intermediate of formula IV, base and the compound of formula V (step (b) above) is preferably performed at sub-ambient temperature, such as any temperature from −30 to 20° C., preferably from −20 to −5° C. (e.g. from −15 to −10° C.).

When the addition of base and the compound of formula V to the reaction mixture is complete, the reaction mixture may be maintained at sub-ambient temperature before being warmed to ambient temperature and worked up (i.e. treated using known techniques such as filtration, evaporation of solvent and/or crystallisation) in order to isolate the product of formula I.

The compound of formula I may then, if desired, be further purified by techniques known to those skilled in the art, such as by methods described in WO 02/083690 and WO 01/028992, the disclosures of which are hereby incorporated by reference (e.g. by recrystallisation from a suitable solvent system, such as isopropanol and water).

Unless otherwise stated, when molar equivalents and stoichiometric ratios are quoted herein with respect to acids and bases, these assume the use of acids and bases that provide or accept only one mole of hydrogen ions per mole of acid or base, respectively. The use of acids and bases having the ability to donate or accept more than one mole of hydrogen ions is contemplated and requires corresponding recalculation of the quoted molar equivalents and stoichiometric ratios. Thus, for example, where the acid employed is diprotic, then only half the molar equivalents will be required compared to when a monoprotic acid is employed. Similarly, the use of a dibasic compound (e.g. Na₂CO₃) requires only half the molar quantity of base to be employed compared to what is necessary where a monobasic compound (e.g. NaHCO₃) is used, and so on.

Advantageously, compounds of formula I obtained via the process of the invention are employed in the preparation of oxabispidines that bear a N-(alkoxy-carbonylamino)alkyl substituent (for example those oxabispidines disclosed in WO 02/083690).

Thus according to a further aspect of the invention, there is provided a process for the preparation of a compound of formula VI,

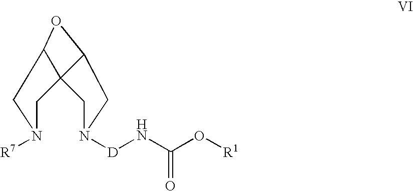

wherein R⁷ represents an amino protective group, such as benzyl, and D and R¹ are as hereinbefore defined, which process comprises a process, as hereinbefore defined for the preparation of a compound of formula I, followed by reaction of that compound with a compound of formula VII,

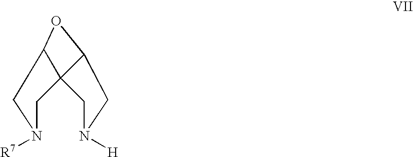

wherein R⁷ is as hereinbefore defined, in the presence of an organic solvent (e.g. toluene).

In this aspect of the invention, reaction between compounds of formula I and VII may be carried out under conditions such as those described in WO 02/083690 (such as at elevated temperature (e.g. 68° C.)).

It will be appreciated by those skilled in the art that, in the processes described above, the functional groups of intermediate compounds may be, or may need to be, protected by protecting groups.

In any event, functional groups which it is desirable to protect include hydroxy and amino. Suitable protecting groups for hydroxy include trialkylsilyl and diarylalkyl-silyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include the amino protective groups mentioned hereinbefore, such as benzyl, sulfonyl (e.g. benzenesulfonyl or 4-nitrobenzene-sulfonyl), tert-butyloxycarbonyl, 9-fluorenylmethoxycarbonyl or benzyloxycarbonyl. The protection and deprotection of functional groups may take place before or after any of the reaction steps described hereinbefore.

Protecting groups may be removed in accordance with techniques which are well known to those skilled in the art and as described hereinafter.

The use of protecting groups is described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

The process of the invention may have the advantage that the compounds of formula I may be produced in a manner that utilises fewer reagents and/or solvents compared to processes disclosed in the prior art.

The process of the invention may also have the advantage that the compound of formula I is produced in higher yield, in higher purity, in less time, in a more convenient (i.e. easy to handle) form, from more convenient (i.e. easy to handle) precursors, at a lower cost and/or with less usage and/or wastage of materials (including reagents and solvents) compared to the procedures disclosed in the prior art.

“Substantially”, when used herein, may mean at least greater than 50%, preferably greater than 75%, for example greater then 95%, and particularly greater than 99%.

The term “relative volume” (rel. vol.), when used herein, refers to the volume (in millilitres) per gram of reagent employed.

The invention is exemplified, but in no way limited, by the following examples.

### EXAMPLE 1

### 2-(tert-Butyloxycarbonylamino)ethyl 2,4,6-trimethylbenzenesulfonate

### Alternative 1

A solution of 2-aminoethanol (40 g, 655 mmol) in dichloromethane (DCM) (320 mL) was heated to 35° C.±3° C. To this, a solution of di-tert-butyl dicarbonate (147.35 g, 655 mmol) in DCM (200 mL) was added over 110 minutes. The reaction mixture was maintained at 35° C.±3° C. during the addition. After the addition was complete, the reaction mixture was maintained at 35° C.±3° C. for one hour. The reaction mixture was then cooled to 22° C.±2° C. and triethylamine (137 mL, 982 mmol) was added in one portion. The reaction mixture was then cooled to −10° C.±3° C. and trimethylamine hydrochloride (31.31 g, 327 mmol) was added in one portion. The resulting mixture was cooled further to −15° C.±3° C. and the reaction mixture was held at this temperature for five minutes. A solution of 2-mesitylenesulfonyl chloride (143.22 g, 655 mmol) in DCM (520 mL) was added slowly enough to maintain the temperature at less than −10° C., (30 minutes). After the addition was complete, the reaction mixture was maintained at −10° C.±3° C. for an additional five minutes. The reaction mixture was warmed to above 0° C. and water (800 mL) was added. The resulting biphasic mixture was stirred rapidly for five minutes and then the phases were separated. The organic layer was concentrated under reduced pressure at a temperature of less than 40° C. and solvent (960 mL) was collected. Isopropanol (960 mL) was added and the resulting solution was concentrated under reduced pressure at a temperature of less than 40° C. and solvent (320 mL) was collected. The resultant solution was cooled to 25° C.±3° C., and water (360 mL) was added slowly, whilst maintaining the temperature at 25° C.±3° C. (This causes the exothermic crystallisation of the title compound.) The mixture was stirred slowly and cooled to 10° C.±3° C., over ten minutes. The product was collected by filtration and then washed by displacement with 1:1 v/v isopropanol:water (160 mL). The product was dried in vacuo at 40° C. for 12±6 hours to give the title compound as a white crystalline solid (186.1 g, 83%).

m.p. 74° C.

¹H-NMR (300 MHz, CDCl₃) δ 6.98 (2H, s), 4.89 (1H, b), 4.01 (2H, t, J=5.1 Hz), 3.39 (2H, q, J=5.3 Hz), 2.62 (6H, s), 2.31 (3H, s), 1.41 (9H, s).

¹H-NMR (300 MHz, DMSO-d₆) δ 7.13 (2H, s), 6.97 (1H, t, J=5.5 Hz), 3.88 (2H, t, J=5.4 Hz), 3.15 (2H, q, J=5.5 Hz), 2.55 (6H, s), 2.29 (3H, s), 1.34 (9H, s).

### Alternative 2

2-Aminoethanol (30.7 kg, 20.501 kmol; 1.0 eq.) was dissolved in dichloromethane (800 L, 1065 kg). The solution was heated to reflux (38° C. to 40° C.). Molten di-tert-butyl dicarbonate (109.6 kg, 0.501 kmol; 1.0 eq.) was added over a period of between 60 and 90 minutes. The reaction mixture was stirred at between 35° C. and 40° C. for 3 hours. The conversion of 2-aminoethanol was checked by GC. When the reaction was complete, the reaction mixture was cooled to 20° C. Triethylamine (105 L, 76.2 kg, 0.75 kmol; 1.50 eq.) was then added to the reaction vessel. The reaction mixture was then cooled to between 0° C. and −5° C. Trimethylamine hydrochloride (35.0 kg, 0.365 kmol; 0.72 eq.) and then a solution of mesitylenesulfonyl chloride (116.5 kg, 0.53 kmol; 1.06 eq.) in dichloromethane (380 L, 507.6 kg) were added to the reaction vessel. This addition was performed slowly enough such that the internal temperature was maintained below −2° C. The reaction mixture was stirred at −5° C. for 30 minutes and conversion was monitored by TLC. The solution was warmed to 3° C., and water (625 L) was added to the reaction mixture and stirring was maintained for between 10 and 20 minutes. After a settling time of between 15 to 30 minutes, the bottom layer (organic layer) was removed. The upper layer (aqueous layer) was discarded. The organic layer was transferred back to the vessel. The solvent was then exchanged from dichloromethane to isopropanol, which was effected by removing solvent (approximately 1000 L of dichloromethane) at reduced pressure (at a maximum temperature of <35° C.) and then replacing it with isopropanol (1050 L). Distillation was then continued until the volume remaining was approximately 590 L, after which water (180 kg) was added to the remaining solvent over 40 minutes at 20° C. The solution was seeded with between 0.6 kg and 0.8 kg of crystalline 2-(tert-butyloxycarbonylamino)ethyl 2,4,6-trimethylbenzenesulfonate. Water (110 kg) was then added over 25 minutes at 20° C., after which crystallisation took place. The resulting suspension was cooled to between 5° C. and 10° C. over 60 minutes, stirred at this temperature for another 60 minutes and then filtered. The product was washed twice with isopropanol:water (1:1 v/v, 220 L) and then dried at a maximum temperature of 35° C. under reduced pressure for 12 hours in a vacuum dryer. This gave the title compound in a yield of 93.8% (161.3 kg).

### EXAMPLE 2

### 3-(tert-Butyloxycarbonylamino)propyl 4-chlorobenzenesulfonate

3-Amino-1-propanol (10 mL, 9.81 g, 130.62 mmol) was dissolved in DCM (78 mL). The resulting mixture was heated to 35° C. and a solution of di-tert-butyl dicarbonate (29.42 g, 130.76 mmol) in DCM (49 mL) was then added over 45 minutes whilst maintaining the temperature at 35° C.±3° C. Once addition was complete, the reaction mixture was stirred at 35° C.±3° C. for a further two hours. The reaction was analysed by TLC (3:1 ethyl acetate:isohexane, potassium permanganate stain). The reaction mixture was cooled to 22° C., and triethylamine (27 mL, 193.71 mmol) was added. After further cooling of the reaction mixture to −10° C., trimethylamine hydrochloride (6.45 g, 66.14 mmol) was added and the temperature reduced to −15° C. Stirring was continued at −15° C. for 5 minutes. A solution of 4-chlorobenzenesulfonyl chloride (27.55 g, 130.53 mmol) in DCM (127 mL) was then added over 45 minutes maintaining the temperature at less than −10° C. Once addition was complete, the reaction was stirred at −10° C. for a further 5 minutes before being warmed to 5° C. over 30 minutes. Water (196 mL) was added and the resulting biphasic mixture stirred rapidly for 5 minutes. The phases were then separated and the upper (aqueous) layer discarded. Solvent (186 mL) was removed by distillation under vacuum, keeping the temperature below 40° C. Propan-2-ol (235 mL) was then added. Further solvent (81 mL) was removed by distillation under vacuum (keeping the temperature below 40° C.), after which the mixture was cooled to 20° C. and water (88 mL) added over 60 minutes to crystallise the product from solution. The product was collected by filtration, washed with 1:1 v/v propan-2-ol:water (100 mL), suction dried as far as possible on the filter, then dried in vacuo (35° C., 16 h) to give the title compound as a white solid (14.42 g, 41.22 mmol, 32%).

¹H NMR (300 MHz, CDCl₃) δ 7.85 (dt, J=8.9, 2.2 Hz, 2H), 7.54 (dt, J=9.0, 2.3 Hz, 2H), 4.61 (s, 1H), 4.13 (t, J=6.2 Hz, 2H), 3.18 (q, J=6.4 Hz, 2H), 1.87 (quintet, J=6.3 Hz, 2H), 1.42 (s, 9H).

¹³C NMR (100 MHz, CDCl₃) δ 155.93 (C═O), 140.56 (aromatic C—H), 134.41 (aromatic C—H), 129.46 (d, J=37.4 Hz, ipso-C), 127.64 (ipso-C), 68.42 (CH₂—O), 36.81 (CH₂—N), 29.35 (—CH₂CH₂CH₂—), 28.32 (C—CH₃).

### ABBREVIATIONS

DCM=dichloromethane
Et=ethyl
eq.=equivalents
GC=gas chromatography
h=hour(s)
Me=methyl
min.=minute(s)
TLC=thin layer chromatography

Prefixes n-, s-, i-, t- and tert- have their usual meanings: normal, secondary, iso, and tertiary.

1. A process for the preparation of a compound of formula I, 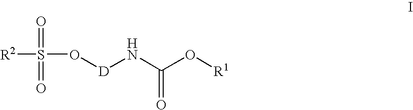
wherein
D represents C₂₋₆ alkylene;
R¹ represents C₁₋₆ alkyl (optionally substituted by one or more substituents selected from —OH, halo, cyano, nitro and aryl), aryl or Het¹;
R² represents unsubstituted C₁₋₄ alkyl, C₁₋₄ perfluoroalkyl or phenyl, which latter group is optionally substituted by one or more substituents selected from C₁₋₆ alkyl, halo, nitro and C₁₋₆ alkoxy;
Het¹ represents a 4- to 14-membered heterocyclic group containing one or more heteroatoms selected from oxygen, nitrogen and/or sulfur, which heterocyclic group may comprise one, two or three rings and may be substituted by one or more substituents selected from oxo, halo, nitro, C₁₋₆ alkyl and C₁₋₆ alkoxy (which latter two groups are optionally substituted by one or more halo atoms); and
wherein each aryl group, unless otherwise specified, is optionally substituted;
provided that D does not represent 1,1-C₂₋₆ alkylene;
which process comprises:
(a) reaction of a compound of formula II,
HO-D-NH₂ II
wherein D is as hereinbefore defined, with a compound of formula III, 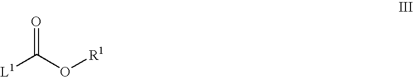
wherein L¹ represents a leaving group and R¹ is as defined above; followed by
(b) reaction of the intermediate of formula IV thereby formed, 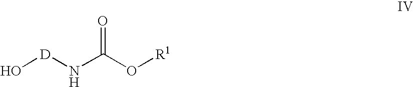
wherein D and R¹ are as defined above, with base and a compound of formula V,
R²S(O)₂L² V
wherein L represents a leaving group and R² is as defined above, and
wherein the intermediate of formula IV is not isolated.

2. A process as claimed in claim 1, wherein D represents —(CH₂)₃— or —(CH₂)₂—.

3. A process as claimed in claim 1, wherein R¹ represents secondary or tertiary C₃₋₅ alkyl.

4. A process as claimed in claim 3, wherein R¹ represents tert-butyl.

5. A process as claimed in claim 1, wherein R² represents phenyl, optionally substituted by one or more substituents selected from methyl, halo and nitro.

6. A process as claimed in claim 6, wherein R² represents 4-chlorophenyl or 2,4,6-trimethylphenyl.

7. A process as claimed in claim 1, wherein L¹ represents —O—C(O)—O-[secondary or tertiary C₃₋₅ alkyl].

8. A process as claimed in claim 7, wherein L¹ represents —O—C(O)—O-tert-butyl.

9. A process as claimed in claim 1, wherein steps (a) and (b) are both carried out in the presence of a solvent that is a C₁₋₂ alkane that is substituted with one or more chloro groups.

10. A process as claimed in claim 9, wherein the solvent is dichloromethane.

11. A process as claimed in claim 10, wherein, after the compound of formula III has been mixed with the aminoalcohol of formula II, the reaction mixture is stirred for a time sufficient to effect dissolution of any oily substance previously formed.

12. A process as claimed in claim 10, wherein step (a) is conducted at a temperature from 32° C. to reflux.

13. A process as claimed in claim 12, wherein, in step (a), a mixture of dichloromethane and the compound of formula II is first heated to a temperature from 32° C. to reflux before reaction is initiated by addition of the compound of formula III.

14. A process as claimed in claim 1, wherein a catalyst is employed to enhance the reactivity of the sulfonating reagent of formula V.

15. A process as claimed in claim 14, wherein the catalyst is trimethylamine, optionally in the form of a hydrochloride salt.

16. A process as claimed in claim 1, wherein the base employed for the reaction between the compounds of formulae IV and V is a tri-(C₁₋₆ alkyl)amine.

17. A process as claimed in claim 16, wherein the base is triethylamine.
